(19) United States
(12) Patent Application Publication
(10) Pub. No.: US 2009/0175964 A1
(43) Pub. Date: Jul. 9, 2009

(54) Composition for enhancing immunity

(75) Inventors: Gautam Banerjee, Bangalore (IN); Jyoti Bhat, Bangalore (IN); Vilas Pandurang Sinkar, Bangalore (IN); Asha Telkar, Bangalore (IN); Smitha Ashok Upadhyaya, Bangalore (IN)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/316,205

(22) Filed: Dec. 10, 2008

(30) Foreign Application Priority Data
Dec. 14, 2007 (IN) 2454/MUM/2007
Apr. 10, 2008 (EP) EP08154305

(51) Int. Cl. A61K 36/00

(52) U.S. Cl. 424/725

(57) ABSTRACT

Disclosed is an edible composition for enhanced immunity comprising theanine or a source of theanine and a herb selected from Shankhpushpi, Shatavari, or a mixture thereof.

### TECHNICAL FIELD OF THE INVENTION

The present invention relates to an edible composition that provides enhanced immunity. The invention more particularly relates to a food composition which comprises a combination of naturally occurring materials which interact synergistically to provide enhanced immunity.

### BACKGROUND OF THE INVENTION

People experience many stresses in their day-to-day life. Such stresses include mental pressures caused by unpleasant interactions at the workplace, in society at large and with the immediate family. Stresses on the human body are also caused by breathing polluted air and consuming contaminated water and food and by exposure to ultra-violet and other radiation. Other sources of stress includes exposure of the human body to continual changes in ambient temperature and other weather conditions. Such continuous exposure of the body to external stresses are believed to cause reduction in the immunity of the human body to diseases, whether of microbiological causes or otherwise. Modern man is on the constant lookout for ways in which his immunity can be enhanced. However, in general, people do not prefer medicinal solutions to solving the problem of reduced immunity. This is because, although medicinal solutions are believed to be effective, they are believed to cause undesirable side-effects. Thus, there is a continuing demand for “natural” solutions to such problems. The science of herbal medicine is one of the ancient sciences, which finds place in modern medical research. Examples of herbal medicine include Ayurveda in India and Traditonal Chinese Medicine in China.

The present inventors have been working on the problem of providing enhanced immunity for individuals using herbs, herbal extracts and other natural materials. One of the problems faced is that although scores of herbs are cited in traditional medicinal system for boosting immunity, there is often a lack of scientific evidence for their efficacy. Further, a large number of combinations of herbs and natural materials have been formulated to form traditional herbal recipes that have been consumed by people over the centuries for various health benefits.

The present inventors have worked extensively to verify the efficacy of the health benefits claimed in the individual herbs and the combination of herbs used in the traditional recipes. During the course of these extensive studies the present inventors determined that a combination of a naturally occurring material usually present in tea viz. theanine when combined with certain herbs viz. shankhpushpi and/or shatavari provides for synergistic benefits in providing immunity. While a large number of combinations were tried by the inventors, it was only these combinations where synergy was observed while combination of theanine with a host of other herbs either provided merely additive benefits or there was antagonistic interaction.

Theanine is known to have many health benefits. These benefits include calming and relaxing effects on the human brain. Theanine is also known to provide indirect anti-microbial effects.

Shankhpushpi is known to have general health benefits. Shatavari has been used in traditional food and medicinal recipes. Shatavari is believed to be a hepatoprotective agent, have positive influence on the gastro-intestinal tract and respiratory system and is also believed to have immunomodulatory effects.

To the knowledge of the present inventors, the combination of theanine with either of the aforementioned two herbs has hitherto not been suggested.

It is an object of the present invention to provide for an edible composition which includes a combination of naturally occurring materials that interact synergistically to provide enhanced immunity benefits.

It is another object of the present invention to provide for an edible food composition that is expected to be consumed by a large cross-section of people whilst providing assured immunity benefits.

### SUMMARY OF THE INVENTION

The present invention provides for an edible composition for enhanced immunity comprising theanine and a herb selected from Shankhpushpi and/or Shatavari.

The present invention also provides a composition comprising theanine and a herb selected from Shankhpushpi and/or Shatavari for use as a medicament in the treatment or prevention of a disease associated with reduced immunity in an individual. The disease may, for example, be cold or influenza.

### DETAILED DESCRIPTION OF THE INVENTION

The invention relates to a composition that provides for enhanced immunity. The composition essentially comprises theanine.

Theanine is a naturally occurring compound and is found mostly in the tea plant. Thus the theanine which is most suitable for use in the edible composition of the invention is derived from tea. Tea is the leaf material from Camellia sinensis var. sinensis or Camellia sinensis var. assamica and material derived therefrom. The tea can be black tea, green tea or oolong tea. In a preferred embodiment the composition comprises tea material and the theanine is present as part of the tea material.

The tea that is produced by complete fermentation and that appears black/brown in color is referred to as black tea. The majority of tea produced is of this kind. Black tea manufacturing technology essentially involves disruption of the cellular integrity of tea shoots, thereby enabling the mixing of substrates (polyphenols) and the enzymes (polyphenol oxidases). This results in the initiation of a series of biochemical and chemical reactions with the uptake of atmospheric oxygen and formation of oxidized polyphenolic compounds that are characteristic of tea along with volatile flavour compounds that impart characteristic aroma to tea.

Green tea is produced by passing the fermentation step. The oxidizing enzymes are killed/denatured by steam blasting the freshly plucked leaf, for example in perforated trays, or by roasting it, for example, in hot iron pans. The steaming is carried out for less than a minute. The leaf is then subjected to further heating and rolling until it turns dark green. The leaves are finally dried to moisture content of 3 to 4 percent. As the fermentation is arrested by the inactivation of polyphenol oxidase, the polyphenols are not oxidized and the leaves remain green. The beverage gives a weaker flavor than black tea due to the absence of the aflavins and the arubigins.

Semi fermented (Oolong) tea is also available. When the fermentation is carried out partially, the resultant teas are referred to as Oolong tea. Oolong tea is mainly consumed for its medical significance.

Theanine has the following structure:

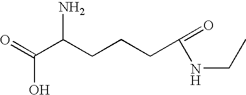

Theanine is known to provide relaxation, calmness and good sleep to the person consuming it. Theanine is found in tea. A 100 ml brew of black tea generally contains about 2.2 millimoles of theanine.

A large number of herbs are listed in traditional herbal medicinal books for immunity benefits. A large number of such herbs are also present in traditional medicinal preparations. However, there is little literature to verify and thereafter quantify the efficacy, if any efficacy is, in fact, determined to be present. The present inventors have evaluated a large number of such herbs and their combinations with theanine using cell culture in-vitro assays to arrive at the combinations which provide the synergistic efficacies. The herbs which are determined to be useful as per the present invention are Shankhpushpi and Shatavari.

Shankhpushpi includes the herbs Evolvulus alsinoides, Convolvulus pluricaulis, and Clitorea turnatea of the family Convolvulaceae. The common name in English is Dwarf morning-glory. It is also sometimes spelt as Shankhapushpi. It is a perennial herb with prostrate branches and small elliptic to oblong leaves, and with flowers which are mostly solitary in upper axils. The whole herb has been used medicinally for fevers, nervous debility, and loss of memory. Shankhapushpi has also been used as a brain tonic. In the present invention any part of Shankhpushpi may be used.

Shatavari (sometimes spelt as Shatawari), also known as Shatamuli includes the herb Asparagus racemosus. It belongs to the family of Liliaceae. The plant is found throughout central and southern India and in lower Himalayas. The herb contains steroidal saponins. It has been traditionally used for making general tonics and is especially used as a tonic for female reproductive function and to increase lactation. Additionally it has been used for providing relief from dysentery, spasms, and also for antioxidant, immunostimulant, and antibacterial benefits. For the purposes of the present invention the preferred parts of Shatavari are tuberous roots.

It is particularly preferred that the herbs are used in the edible composition of the invention in the form of aqueous extracts. A typical process of water extraction involves boiling the herb or the desired part of the herb in water in a weight ratio of about 1:8 for about 1 hour. The extract thus obtained is then dried to a moisture content of less than about 8.0% to obtain a powder. In the present invention, the herbs are preferably present as powders of the water extracts of the various herbs.

While edible food compositions are more preferred forms of delivering the edible composition of the invention, the edible composition may also be delivered to the consumer in generally accepted pharmaceutical forms e.g. as a tablet, capsule, powder or syrup form.

The edible composition is ideally delivered in a food product. While any food product may deliver the edible composition of the invention, suitable food products are tea beverages, coffee beverages, fruit juices, vegetable purees, soup, jams, spreads and sauces.

The food product preferably comprises the theanine and the herb in a dry weight ratio in the range of 50:1 to 1:5.

The composition is preferably used to treat or prevent a disease associated with reduced immunity in an individual. The disease may, for example, be cold or influenza. Preferably the composition is administered to the individual orally.

The composition may also be used in the manufacture of a medicament for the treatment or prevention of the disease associated with reduced immunity in an individual.

### Source and Origin of the Ingredients Used in Developing the Invention

| | | Source and geographical origin |
|---|---|---|
| Ingredient | Form of ingredient | of ingredient |
| Theanine | Dry powder | Tayo, Japan under the Sun- |
| | | Theanine ™ brand. |
| Shankhpushpi | Water extract powder | Indo World Trading Corporation |
| | | New Delhi (India) |
| Shatavari | Water extract powder | Indo World Trading Corporation |
| | | New Delhi (India) |

### EXAMPLES

The invention will now be demonstrated with examples. The examples are by way of illustration only and do not limit the scope of invention in any manner.

### Evaluation of the Immunological Efficacy of Herbs Using Activation of Macrophage in In-Vitro Cell Culture Assays

The procedure to conduct the cell culture assays for the activation of macrophage is described below:

### Materials Used:

Raw 264.7 cell line were obtained from National Centre for Cellular Sciences (NCCS) Pune, India.

DMEM (Dulbecos Modified Eagle Medium) RPMI (Rosewell Park Memorial Institute) medium, Fetal Bovine Serum (FBS), Phorbol-12-Myristate-13-acetate (PMA) were obtained from Sigma-Aldrich, U.S.A.

Latex bead (3 μm) was procured from Polysciences Inc, Germany. Geimsa stain was obtained from S.D. Fine Chem. Ltd, India

All other reagents were of analytical grade.

### Methods:

### A. Cell Culture

Raw 264.7 cell line was continuously maintained in DMEM with 10% FCS (Foetal calf serum). They were subcultured every 2-3 days and incubated at 37° C. at 5% carbon dioxide atmosphere.

B. Priming Macrophage Cell Line with Plant Extracts

### (i) Preparation of Plant Extract

Desired plant material (200 mg) was weighed into 5 ml of Distilled water and kept on a rotator for 2 hrs at room temperature. This was boiled at 80° C. for 1 hr and the volume was made up to 10 ml. The supernatant was filtered through 0.22 μm filter and used for further analysis.

### (ii) Macrophage Engulfment Assay

Raw 264.7 cells were plated on coverslip at 10⁵ cell/cover glass and allowed to adhere. The cells were incubated with 100 ng/ml PMA (10 mins) or plant extract, theanine or the combination of plant extract and theanine for 2 hrs. Post incubation, the cells were incubated for 2 hrs with 106 latex beads/10⁵ cells (Cell: beads=1:10). The cells on the coverslip were air dried and fixed with methanol. Cells were stained with Giemsa for 10 mins and counted microscopically.

The results on macrophage engulfment assay were expressed as Phagocytosis index i.e. the total number of beads within the cells per 100 cells.

The experiments (four samples per experiment) were conducted using the materials at the concentration indicated in Table 1. The data on the mean phagocytosis index, is also summarized in Table 1 along with the standard deviation. The data analysed using statistical methods indicates greater than 95% confidence in the t-test.

TABLE 1

| | Concentration, | Phagocytosis |
|---|---|---|
| Active(s) | wt % in medium | index |
| Theanine | 0.01 | 1.38 ± 0.08 |
| Theanine | 0.015 | 1.45 ± 0.50 |
| Theanine | 0.02 | 1.40 ± 0.39 |
| Shankhpushpi | 0.011 | 1.4 ± 0.18 |
| Shankhpushpi | 0.001 | 1.33 ± 0.05 |
| Shatavari | 0.005 | 1.3 ± 0.37 |
| Shatavari | 0.015 | 1.4 ± 0.2 |
| Theanine + | 0.01 + 0.001 | 1.69 ± 0.09 |
| Shankhpushpi | | |
| Theanine + | 0.01 + 0.005 | 2.1 ± 0.5 |
| Shatavari | | |

The data in Table 1 indicates that any one of the above actives viz. theanine, shatavari and shankhpushpi over a wide range of concentrations (0.001 to 0.02) gives a phagocytosis index in the range of 1.3 to 1.45. However a combination of theanine with the herbs shankhpushpi or shatavari provides for a significantly higher phagocytosis index than that achievable with the individual herbs in the same concentration range.

The invention therefore provides for an edible composition which includes a combination of naturally occurring materials that interact synergistically to provide enhanced immunity benefits.

1. An edible composition for enhanced immunity comprising theanine and a herb selected from Shankhpushpi, Shatavari, or a mixture thereof.

2. An edible composition as claimed in claim 1 comprising aqueous extract of said herb.

3. An edible composition as claimed in claim 1 wherein the theanine is derived from tea.

4. An edible composition as claimed in claim 3 wherein said tea is black tea, green tea or oolong tea.

5. An edible composition as claimed in claim 3 wherein the theanine is present in the composition as part of tea material.

6. An edible composition as claimed in claim 1 wherein the composition is a tablet, capsule, powder or syrup form.

7. An edible composition as claimed in claim 1 wherein the edible composition is a food product.

8. An edible composition as claimed in claim 7 wherein said food product is a tea beverage, a coffee beverage, a fruit juice, a vegetable puree, a soup, a jam, a spread or a sauce.

9. An edible composition as claimed in claim 1 wherein the theanine and the herb are present in a dry weight ratio in the range of 50:1 to 1:5.

10. A composition comprising theanine and a herb selected from Shankhpushpi, Shatavari, or a mixture thereof for use as a medicament in preventing or treating a disease related to reduced immunity in an individual.

11. Use of a composition comprising theanine and a herb selected from Shankhpushpi, Shatavari, or a mixture thereof in the manufacture of a medicament for preventing or treating a disease related to reduced immunity in an individual.

12. A method of preventing or treating a disease related to reduced immunity in an individual, the method comprising administering to the individual a composition comprising theanine and a herb selected from Shankhpushpi, Shatavari, or a mixture thereof.

13. Composition, use or method as claimed in claim 1 wherein the disease is cold and/or influenza.

14. Composition, use or method as claimed in claim 1 wherein the composition is edible and is administered orally to the individual.
